(19) United States
(12) Patent Application Publication
(10) Pub. No.: US 2026/0174815 A1
(43) Pub. Date: Jun. 25, 2026

(54) FEED ADDITIVE FOR IMPROVING HEALTH AND PERFORMANCE OF FARMING ANIMALS

(71) Applicant: Nutreco IP Assets B.V., Boxmeer (NL)

(72) Inventors: David Manuel BRAVO, Boxmeer (NL); Karin BERGER BÜTER, Boxmeer (NL)

(21) Appl. No.: 19/537,351

(22) Filed: Feb. 11, 2026

Related U.S. Application Data
(63) Continuation of application No. PCT/EP2024/072925, filed on Aug. 14, 2024.

(30) Foreign Application Priority Data
Aug. 14, 2023 (EP) 23191357.5

(51) Int. Cl. A61K 36/185; A23K 10/30; A61K 8/9789; A61K 36/899

(52) U.S. Cl. CPC A61K 36/185; A23K 10/30; A61K 8/9789; A61K 36/899; A61K 2300/00

(57) ABSTRACT

The present invention is in the field of feed additives for animals, e.g., farming animals, in particular feed additives to be added to animal feed to effect an improved performance, e.g., an increasing feed intake, increased average daily gain, increased feed efficiency (i.e., decreased feed conversion ratio), and/or increased milk yield, of such animals.

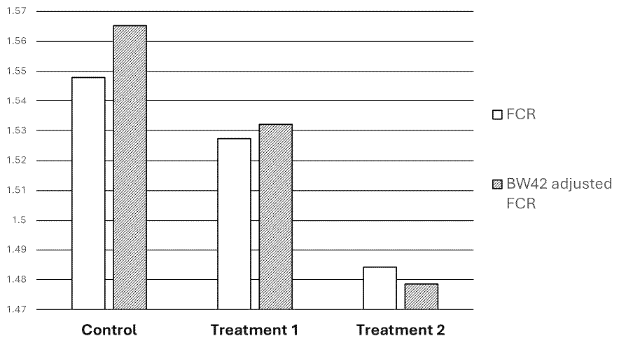

### CROSS REFERENCE TO RELATED APPLICATIONS

The present application is a continuation of International Application No. PCT/EP2024/072925, filed Aug. 14, 2024, which claims priority to European Patent Application No. 23191357.5, filed Aug. 14, 2023, both of which are hereby incorporated by reference herein in their entireties.

### FIELD OF THE INVENTION

The present invention is in the field of feed additives for use in animal feed, in particular for use in preventing and/or reducing inflammation in animals, such as for use in preventing and/or reducing microbial infection and/or diseases caused by such microbial infection in animals.

### BACKGROUND OF THE INVENTION

Inflammation is necessary for life. It is the first step in the healing or repair process that helps the body fight off bacteria, other pathogens and their toxins, and repair damaged tissue. While inflammation is necessary, there is a trade-off in terms of animal performance. Hence, livestock and poultry producers need to pay close attention to inflammation and the immune system.

When a pathogen or bacteria crosses one of the body's barriers, such as the skin, mucous membrane, or blood vessel linings, the immune system will detect the invasion. The immune system then sends signals to cytokines, pro-inflammatory proteins, indicating that help is needed. This initiates the movement of white blood cells toward the site of inflammation, infection or trauma. The first responders to the site are white blood cells called phagocytes. These cells help protect the body by ingesting harmful foreign particles, bacteria, and dead or dying cells. There are two forms of phagocytes. Neutrophils are small, granular leukocytes that quickly appear at the site of a wound and ingest bacteria. Monocytes are larger leukocytes that appear about 3 days after infection and scavenge for bacteria, foreign particles and dead cellular material left behind by the neutrophils. The neutrophils appear at the site first and work to engulf and destroy the pathogen or bacteria. They then display pieces of the pathogens on their surface to signal the monocytes to help continue the attack on the invading pathogens. There are also a group of anti-inflammatory cytokines that help control the pro-inflammatory cytokine response.

In animal husbandry, inflammation reduces profitability and product quality and endangers the health of animals. Inflammation can occur under all animal husbandry systems, including in intensive, modern animal production systems. Inflammation costs energy. Raising the body temperature by just a degree results in 13% energy loss. In smaller animals like poultry, a small increase still comes with a significant energy expenditure and any energy use comes at the cost of feed conversion, which in turn affects the cost of production. When faced with inflammation, animals' bodies re-prioritize energy utilization, and the immune response is prioritized before growth. When animals experience a shortage of energy due to increased exertion from inflammation, they cannot reach their full genetic potential regarding growth, meat yield, milk yield or eggs. Hence, controlling inflammation increases the likelihood of a positive ROI regarding feed conversion.

Additionally, inflammation affects the integrity of the intestinal walls. The effects can range from improperly sealed tight junctions, over-reduced mucus, loss of gut structure (decreased villi length and crypt depth) and even open lesions. This weakened integrity of the intestinal lining can make it easier for pathogens such as Enterococcus cecorum or Campylobacter to translocate past the epithelial wall. While such pathogens are often present in broilers, they can become a severe problem when they translocate. Additional substances such as mycotoxins or contaminants tend to infect the animal at a higher rate when inflammation has weakened the structures.

It is an object of the present invention to provide a composition that has anti-inflammatory properties and/or that may increase feed intake, average daily gain, feed efficiency, and/or milk yield in animals, particularly farming animals, and/or that has antimicrobial activity, particularly against Gram positive and Gram negative bacteria, more particularly against Vibrio parahaemolyticus and/or Enterococcus cecorum.

### SUMMARY OF THE INVENTION

The present disclosure provides a composition comprising Glycyrrhiza glabra, or an extract thereof and at least one of Satureja hortensis, or an extract thereof; and Artemisia vulgaris, or an extract thereof; said composition preferably comprising Glycyrrhiza glabra aerial parts, e.g., Glycyrrhiza glabra leaves, or an extract thereof, and at least one of i) Satureja hortensis aerial parts, or an extract thereof; and ii) Artemisia vulgaris aerial parts, or an extract thereof. The Satureja hortensis aerial parts, the Artemisia vulgaris aerial parts, and/or the Glycyrrhiza glabra aerial parts, e.g., Glycyrrhiza glabra leaves, may be dried, and may optionally be ground.

The extract may be selected from the group consisting of an aqueous extract, ethanolic extract, methanolic extract, isopropanolic extract, ethylacetate extract, acetonic extract, hexane extract, a supercritical CO2 extract, or a combination of any of these.

The present disclosure also relates to an animal feed comprising a composition as taught herein. The composition may be incorporated into the animal feed in an amount of about 5 to about 10000 mg/kg feed.

The present disclosure further pertains to use of a composition as taught herein, a feed additive as taught herein, or an animal feed as taught herein for feeding animals and/or for improving performance of animals. In an embodiment, the composition as taught herein, feed additive as taught herein, or animal feed taught herein may be used for increasing feed intake, increasing average daily gain, increasing feed efficiency, and/or increasing milk yield.

The present disclosure further provides a composition as taught herein, a feed additive as taught herein, or an animal feed as taught herein for use in decreasing IL6 levels in an animal.

The present disclosure also teaches a composition as taught herein, a feed additive as taught herein, or an animal feed as taught herein for use in preventing and/or reducing inflammation.

The present disclosure is further concerned with a composition as taught herein, a feed additive as taught herein, or an animal feed as taught herein for preventing and/or reducing microbial infection and/or diseases causes by such microbial infection.

In an embodiment, the microbial infection is caused by a microbe selected from the group consisting of a Gram-negative and/or a Gram-positive bacterium.

In an embodiment, the causative microbe is a Vibrio species, such as Vibrio parahaemolyticus.

In another embodiment, the causative microbe is an Enterococcus species, such as Enterococcus cecorum.

The extract may be selected from the group consisting of an aqueous extract, ethanolic extract, methanolic extract, isopropanolic extract, ethylacetate extract, acetonic extract, hexane extract, or a supercritical CO2 extract, or a mixture of any of these.

The feed additive may be intended for inclusion in feed for farming animals or companion animals.

The farming animals may be selected from the group consisting of poultry, swine, ruminants, e.g., beef cattle and dairy cattle, fish, e.g. salmon, trout, seabream, sea brass, tilapia, tuna, and the like, and crustaceans, e.g., shrimp. The companion animals may be selected from the group consisting of ornamental fish, cats, dogs, horses, rabbits, guinea pigs, and hamsters.

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references cited herein are incorporated herein in their entirety by reference.

The term “aerial part” when referring to a plant refers to anything that exists in the air or in the space above a solid surface. The aerial part of plants simply denotes the structures of a plant that are above ground, including the stems, leaves, petioles, flowers, fruits and seeds. The term includes any aerial part alone, e.g., leaves, or stem, or the entire aerial part (i.e., all plant parts above the ground).

The term “leaves” when referring to a plant refers to any usually flattened green outgrowth from the stem of a vascular plant. As the primary sites of photosynthesis, leaves manufacture food for plants, which in turn ultimately nourish and sustain all land animals. Botanically, leaves are an integral part of the stem system. They are attached by a continuous vascular system to the rest of the plant so that free exchange of nutrients, water, and end products of photosynthesis (oxygen and carbohydrates in particular) can be carried to its various parts.

The term ‘farming animal’ refers to animals that are kept or raised for agricultural purposes, like for consumption or to generate income by for example meat, eggs or milk products. Farming animals can be grouped based on their digestive system. Such groups include monogastric, ruminant and pseudo-ruminant animals.

The term “pellets” or “feed pellets” as used herein refers to small particles or a body typically created by compressing an original material, for instance a mixture of raw feed material, typically fermentable feed ingredients such as grains, cereals, legumes, roughage, and the likes. Feed pellets may also comprise other feed ingredients such as meat meal, fish meal, bone meal, by-process products, oil, fat, fillers or any mixture thereof, etc., as well as minerals, vitamins and trace elements and others. Animal feed pellets vary in their composition as well as structural properties (e.g. hardness, density, durability, shape, size, etc.) depending on the nutritional needs, eating habits, digestive system (monogastric system, ruminant digestive system, etc.) and habitat (e.g., aquatic, terrestrial, domestic, etc.) of the animal for which the feed pellet is intended. Animal feed pellet may have any size, shape (e.g. round, rectangular, cylindrical, etc), weight and/or length. It is understood that the weight of the feed pellet will depend on the feed pellet composition per se (e.g. some ingredients have a greater weight or density than others) as well as the shape, size and length of the finished feed pellet product. It is known that the size, shape, weight and/or length of the feed pellet will influence pellet durability. This is true for any method for making feed pellets, including method as taught herein. It is further commonly agreed in the field of agriculture and animal nutrition that animals (e.g. young and adult livestock animals like beef (e.g. beef calf), dairy cows (e.g. dairy calf), and pigs, etc.) benefit more or make better gains (e.g. weight gain, increased height, enhanced growth curve) on pelleted feed than a meal ration because pelleted feed is in a more concentrated, readily edible and palatable form than meal or mash ration. Pelleted feed has been shown to ease food intake and minimize feed waste during the eating process. It was shown that most animals, if given the choice between the same feed in a pellet or a mash form, will prefer the pellet form. Animal feed pellets are typically produced on an industrial scale using for example a pelleting process. The skilled person is well-acquainted with processes for producing animal feed pellets.

The term “secondary plant constituents” as used herein refers to specialized compounds present in plants that do not aid in the growth and development of plants but are required for the plant to survive in its environment. Such secondary plant constituents may be essential for communicating with other organisms in mutualistic (e.g. attraction of beneficial organisms such as pollinators) or antagonistic interactions (e.g. deterrent against herbivores and pathogens). They may further assist in coping with abiotic stress such as increased UV-radiation. The broad functional spectrum of specialized metabolism is still not fully understood.

The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

### DETAILED DESCRIPTION OF THE INVENTION

This patent application has been drafted into sections. However, these sections should not be read in isolation. Unless otherwise specified, each section is to be read in combination with the other sections. The various optional and preferred features can also be combined, even when taken from different parts of the specification. Likewise, all “aspects” and “embodiments” can be combined. No separation of embodiments is intended, unless explicitly stated.

The present inventors have surprisingly found that a composition comprising an extract of Glycyrrhiza glabra leaves and at least one of an extract of Satureja hortensis aerial parts, and an extract of Artemisia vulgaris aerial parts, produced a synergistic effect on mitigating inflammation in vitro. This effect is larger than the effect of the individual plants, and even more pronounced when extracts of all three plants together were used.

Moreover, the present inventors have surprisingly found that inclusion of a composition as taught herein in a relatively small amount in broiler feed or salmon feed, increased their performance in terms of body weight gain, average daily gain, feed intake, and/or feed efficiency.

Further, it was found that inclusion of a composition as taught herein preventing and/or reducing microbial infection and/or diseases caused by such microbial infection in an animal.

Satureja hortensis (aka summer savory) is an annual herbaceous crop species, strongly branched, with linear leaves belonging to the Lamiaceae family. Artemisia vulgaris L. (aka common mugwort) is a plant from the Artemisia genus with a widespread distribution (Europe, Asia, North and South America, and Africa). Glycyrrhiza glabra is a Fabaceae plant from the Leguminosae family, native to Eurasia, in central and south-western Asia and the Mediterranean region. The roots of Glycyrrhiza glabra are known as licorice whereas its leaves are considered an agricultural waste.

Composition, Animal Feed and/or Feed Additive

Thus, the present disclosure provides a composition comprising Glycyrrhiza glabra, or an extract thereof and at least one of i) Satureja hortensis, or an extract thereof; ii) Artemisia vulgaris, or an extract thereof. Preferably the composition comprises Glycyrrhiza glabra aerial parts, even more preferably Glycyrrhiza glabra leaves, or an extract thereof, and at least one of i) Satureja hortensis aerial parts, or an extract thereof; and ii) Artemisia vulgaris aerial parts, or an extract thereof.

In an embodiment, the composition taught herein comprises Glycyrrhiza glabra, or an extract thereof and Satureja hortensis, or an extract thereof, preferably Glycyrrhiza glabra aerial parts, even more preferably Glycyrrhiza glabra leaves, or an extract thereof, and Satureja hortensis aerial parts, or an extract thereof. Said composition may optionally further comprise Artemisia vulgaris aerial parts, or an extract thereof.

In an embodiment, the composition taught herein comprises Glycyrrhiza glabra, or an extract thereof, and Artemisia vulgaris, or an extract thereof, preferably Glycyrrhiza glabra aerial parts, even more preferably Glycyrrhiza glabra leaves, or an extract thereof, and Artemisia vulgaris aerial parts, or an extract thereof. Said composition may optionally further comprise Satureja hortensis aerial parts, or an extract thereof.

In a preferred embodiment, the composition taught herein comprises Glycyrrhiza glabra aerial parts, or Glycyrrhiza glabra leaves, Satureja hortensis aerial parts, and Artemisia vulgaris aerial parts.

In another embodiment, the composition taught herein comprises an extract of Glycyrrhiza glabra aerial parts or Glycyrrhiza glabra leaves, an extract of Satureja hortensis aerial parts, and an extract of Artemisia vulgaris aerial parts.

The composition may comprise the various plants or plant parts, or plant extracts in any ratio. For example, the composition may comprise 5-95 wt %, such as 10-90 wt %, or 15-85 wt %, 20-80 wt %, 25-75 wt %, 30-70 wt %, 35-65 wt %, 40-60 wt %, or 45-55 wt %, Glycyrrhiza glabra aerial parts, or Glycyrrhiza glabra leaves, preferably dried and ground, 5-95 wt %, such as 10-90 wt %, or 15-85 wt %, 20-80 wt %, 25-75 wt %, 30-70 wt %, 35-65 wt %, 40-60 wt %, or 45-55 wt %, Artemisia vulgaris aerial parts, preferably dried and ground, and 5-95 wt %, such as 10-90 wt %, or 15-85 wt %, 20-80 wt %, 25-75 wt %, 30-70 wt %, 35-65 wt %, 40-60 wt %, or 45-55 wt %, Satureja hortensis aerial parts, preferably dried and ground.

The aerial parts, e.g., leaves, may be used fresh or may be dried by any means known in the art. The skilled person knows suitable drying technologies. For example, drying may take place using a drum drier, belt dryer, dehydrator drying, vacuum drying, microwave drying, or infrared-drying, or natural drying (sun drying). Thus, the composition taught herein may comprise dried Satureja hortensis aerial parts, Artemisia vulgaris aerial parts, and/or Glycyrrhiza glabra aerial parts, even more preferably Glycyrrhiza glabra leaves. The skilled person is capable of determining a suitable degree of drying.

Prior to or after drying, preferably after drying, the aerial parts, e.g., leaves, may be ground to any suitable size for inclusion in animal feed. For example, the fresh or dried aerial parts, e.g., leaves, may be ground to an average particle size of smaller than 2 mm, preferably smaller than 1.5 mm, even more preferably smaller than 1 mm, yet more preferably smaller than 0.8 mm, even more preferably smaller 0.6 mm, most preferably smaller than 0.5 mm.

The composition may comprise aerial parts or leaves of all plants or may comprise aerial parts or leaves of one or two plants, whilst it further comprises an extract of the other one or two plants contained therein. Alternatively, the composition may comprise an extract of all three plants.

The Satureja hortensis, Artemis vulgaris, and Glycyrrhiza glabra that may be used in the composition taught herein may be any variety of the respective plant species.

The composition may comprise aerial parts, optionally ground and/or optionally dried, of the plants, or may comprise an extract of all plants, or may comprise an extract of one plant and aerial parts or leaves of the other plant or plants or may comprise an extract of two of the plants and aerial parts or leaves of the other plant. In a preferred embodiment, the composition taught herein comprises aerial parts or leaves of all plants used.

The composition may comprise additional components such as inert fillers.

The composition taught herein may be included in animal feed in an amount of about 1 mg/kg to about 10000 mg/kg of feed, preferably about 5 mg/kg to about 8000 mg/kg of feed, more preferably about 10 mg/kg to about 6000 mg/kg of feed, even more preferably about 15 mg/kg to about 4000 mg/kg of feed, yet more preferably about 20 mg/kg to about 3000 mg/kg of feed, again more preferably about 25 mg/kg to about 2000 mg/kg of feed, such as about 30 mg/kg to about 1500 mg/kg of feed, about 40 mg/kg to about 1200 mg/kg of feed, about 50 mg/kg to about 1000 mg/kg of feed, or about 60 mg/kg to about 800 mg/kg of feed.

Alternatively, or additionally, an equivalent amount of extract of the aerial parts or leaves of the respective plants may be used. Plant extraction is a process that aims to extract certain components (so-called secondary plant constituents, including alkaloids, terpenoids, saponins, phenolic compounds, flavonoids, and/or tannins) present in plants. It is a solid/liquid separation operation: a solid object (in the present case aerial parts or leaves of one or more of the plants of the composition taught herein, optionally dried and/or ground) is placed in contact with a fluid (the solvent). The plant components of interest are then solubilised and contained within the solvent. The solution thus obtained is the desired extract. The solvent may eventually be eliminated, although this is not required. The solvent may be any solvent suitable for preparing plant extracts for use in animal feed. Suitable solvents include, without limitation, polar solvents (e.g., water, alcohols such as ethanol, methanol and isopropanol), intermediate polar solvent (e.g., acetone, dichloromethane), and nonpolar solvents (e.g., ethylacetate, hexane, ether, chloroform). The skilled person may select a single solvent or two or more solvents to prepare an extract in accordance with the present disclosure. In general, extraction procedures include maceration, digestion, decoction, infusion, percolation, Soxhlet extraction, superficial extraction, ultrasound-assisted, and microwave-assisted extractions, supercritical fluid extraction (e.g. with supercritical carbon dioxide), accelerated solvent extraction, and solid-phase extraction. Fractionation and purification of phytochemical substances may be achieved through application of various techniques such as liquid-liquid partitioning, cross-flow filtration techniques and chromatographic methods, such as paper chromatography, thin-layer chromatography, gas chromatography, and high-performance liquid chromatography, centrifugal partitioning chromatography. Finally, compounds obtained are characterized using diverse identification techniques such as mass spectroscopy, infrared spectroscopy, ultraviolet spectroscopy, and nuclear magnetic resonance spectroscopy, or any combination thereof. The skilled person is capable of selecting a suitable extraction method.

The extract may suitably be an extract prepared using a polar solvent or a mixture of polar solvents, such as a water-ethanol mixture.

The present disclosure further provides a feed additive comprising the composition taught herein. Moreover, the present disclosure provides use of the composition taught herein as a feed additive.

Said feed additive may further comprise vitamins, minerals, organic acids, antioxidants, and/or pigments. Said feed additive may be provided in the form a premix. The feed additive is preferably intended for inclusion in feed for farming animals or companion animals.

Said feed additive may be supplied with written instructions to include it into animal feed in an amount of the composition taught herein of about 1 mg/kg to about 10000 mg/kg of feed, preferably about 5 mg/kg to about 8000 mg/kg of feed, more preferably about 10 mg/kg to about 6000 mg/kg of feed, even more preferably about 15 mg/kg to about 4000 mg/kg of feed, yet more preferably about 20 mg/kg to about 3000 mg/kg of feed, again more preferably about 25 mg/kg to about 2000 mg/kg of feed, such as about 30 mg/kg to about 1500 mg/kg of feed, about 40 mg/kg to about 1200 mg/kg of feed, about 50 mg/kg to about 1000 mg/kg of feed, or about 60 mg/kg to about 800 mg/kg of feed, or, in case an extract of one or more of the plants is used, an equivalent amount of extract.

The present disclosure also provides an animal feed comprising a composition as taught herein.

The animal feed may be in any form suitable for ingestion by the relevant animal. For example, poultry feed may be in the form of crumble, mash, or pellets. Swine feed may be in the form of meal or pellets. Feed for fish and crustaceans is usually in the form of extruded or pressed pellets but may also be in the form of flakes. In an embodiment, the animal feed is in the form of a crumble, mash, meal, flakes, or pellets, which pellets may be extruded or may be pressed.

The animal feed generally comprises protein, fat, carbohydrates, minerals and vitamins. It may further comprise organic acids, and other feed additives. The skilled person knows how to formulate a suitable animal feed depending on the target animal.

The amount of extract or extracts to be incorporated into the animal feed and/or feed additive may be determined by equating the content of one or more secondary plant constituents in the extract to those in the respective plant parts. An amount of extract that provides a content of one or more secondary plant constituents that is equivalent to the content of said one or more secondary plant constituents in the indicated amount of aerial parts or leaves may thus be incorporated into said animal feed.

In an embodiment, the extract is selected from the group consisting of an aqueous extract, ethanolic extract, methanolic extract, isopropanolic extract, ethylacetate extract, acetonic extract, hexane extract, or a supercritical CO2 extract, or a mixture of any of these. Alternatively, a mixture of solvents may be used for extraction, as set forth above.

Methods of Using the Composition, Animal Feed and/or Feed Additive

In an aspect, the present disclosure provides a method of feeding an animal, e.g., a farming animal or a companion animal, with a composition, a feed additive, or an animal feed as taught herein.

In a further aspect, the present disclosure relates to use of a composition as taught herein, a feed additive as taught herein, or an animal feed as taught herein for increasing or improving performance of an animal, preferably a farming animal. The disclosure is also concerned with a method of increasing or improving performance of an animal, preferably a farming animal, comprising administering to said animal a composition, a feed additive, or an animal feed as taught herein.

In another aspect, the present disclosure is concerned with use of a composition as taught herein, a feed additive as taught herein, or an animal feed as taught herein for increasing feed intake, increasing average daily gain, increasing feed efficiency (i.e., decreasing feed conversion ratio), and/or increasing milk yield. The disclosure also concerns a method of increasing feed intake, increasing average daily gain, increasing feed efficiency (i.e., decreasing feed conversion ratio), and/or increasing milk yield, said method comprising administering to said animal a composition, a feed additive, or an animal feed as taught herein.

In yet another aspect, the present disclosure provides use of a composition as taught herein a feed additive as taught herein, or an animal feed as taught herein, for use in decreasing IL6 levels in an animal.

The present disclosure also provides use of a composition as taught herein, a feed additive as taught herein, or an animal feed as taught herein for use in preventing and/or reducing inflammation in an animal.

The present disclosure further provides a method of improving performance of animals, e.g., increasing feed intake, increasing average daily gain, increasing feed efficiency (i.e., decreasing feed conversion ratio), and/or increasing milk yield, said method comprising the step of administering to said animals a composition as taught herein, a feed additive as taught herein, or an animal feed as taught herein.

The present disclosure also provide a composition as taught herein, a feed additive as taught herein, or an animal feed as taught herein for use as a medicament.

Moreover, the present disclosure provides a method of decreasing IL6 levels in an animal, said method comprising the step of administering a composition as taught herein, a feed additive as taught herein, or an animal feed as taught herein to said animal.

Also, the present disclosure provides a method of preventing, alleviating the severity of, and/or reducing inflammation in an animal, said method comprising the step of administering a composition as taught herein, a feed additive as taught herein, or an animal feed as taught herein to said animal.

Further, the present disclosure provides a composition as taught herein, a feed additive as taught herein, or an animal feed as taught herein for use in reducing mortality, or a method of reducing mortality in an animal comprising administering to the animal a composition, feed additive, or animal feed as taught herein, e.g., in disease challenged animals, e.g. in shrimp challenged with a disease, e.g., challenged with acute hepatopancreatic necrosis disease (AHPND), e.g., by bacteria of the Vibrio genus, e.g. of the species Vibrio parahaemolyticus.

The present disclosure further provides use of a composition as taught herein, a feed additive as taught herein, or an animal feed as taught herein for use in preventing, alleviating the severity of, and/or reducing microbial infection and/or diseases caused by such microbial infection.

The present disclosure further relates to a method of preventing, alleviating the severity of, and/or reducing microbial infection and/or diseases caused by a microbial infection, said method comprising administering a composition as taught herein, a feed additive as taught herein, or an animal feed as taught herein to said animal.

The microbial infection may be caused by any microbe, e.g., a fungus, a yeast, a protozoa, a Gram-positive bacterium, or a Gram-negative bacterium. The microbial infection may, e.g., be caused by Gram positive or Gram negative bacteria.

The microbial infection may, e.g., be caused by Gram positive or Gram negative bacteria. Non-limiting examples of bacteria that may be causing the microbial infection referred to herein include Escherichia species, Salmonella species, Vibrio species, Flavobacterium species, Campylobacter species, Clostridium species, Streptococcus species, Piscirickettsia species such as Piscirickettsia salmonis causing Salmon Rickettsial Syndrome (SRS), and others, such as Escherichia coli, Salmonella typhimurium, Salmonella dublin, Salmonella enterica, Salmonella enteritidis, Clostridium perfringens, Pasteurella multocida, Listeria monocytogenes, Mycoplasma bovis, Haemophilus somnus, Campylobacter jejunum, Campylobacter hepaticus, Campylobacter bilis, and the like.

In an embodiment, the microbial infection is caused by a Gram-negative bacteria, e.g., of the genus Flavobacterium or of the genus Vibrio.

Flavobacteriosis is a group of three major diseases caused by members of the Flavobacteriaceae, mainly Flavobacterium branchiophilum, F. columnare, F. psychrophilum, and Tenacibaculum maritimum. Columnaris disease or infection (myxobacterial disease, saddleback, fin rot, and cotton wool disease) is caused by F. columnare (freshwater) and T. maritimum (marine water). Bacterial cold-water disease (peduncle disease, rainbow trout fry syndrome) is caused by F. psychrophilum, and bacterial gill disease (BGD) or proliferative gill disease (PGD) is caused by F. branchiophilum (Sie-Maen Chong. Aquaculture Pathophysiology. Volume I. Finfish Diseases 2022, Chapter 28, Pages 367-377). Flavobacterium columnare is one of the oldest known diseases among fish and manifests itself as an infection commonly known as columnaris. It may infect many different wild and cultured freshwater fish species, such as (but not limited to) carp, channel catfish, goldfish, eel, perch, salmonids and tilapia. F. columnare causes acute to chronic infections and typically affects the gills, the skin and fins. In an embodiment of the present disclosure, the microbial infection is caused by a microbe of the species F. columnare. The infected animal, or the animal at risk of infection may be a freshwater fish species, e.g., selected from the group consisting of carp, channel catfish, goldfish, eel, perch, salmonids and tilapia.

In an embodiment, the bacterium of the genus Flavobacterium is selected from the group consisting of F. columnaris, F. johnsoniae, Tenacibaculum maritimum (formerly known as Flexibacter maritimus), F. psychrophilum, F. branchiophilum, Tenacibaculum ovolyticum (formerly Flexibacter ovolyticus), and Chryseobacterium scopthalmum (formerly F. scopthalmum).

In an embodiment, the microbial infection is caused by a Gram-negative bacteria, e.g., of the genus Vibrio.

In aquaculture, several Vibrio spp. are currently considered pathogens or opportunistic pathogens of reared finfish, shellfish, and shrimp, among which V. anguillarum, V. ordalii, V. vulnificus, V. alginolyticus, V. parahaemolyticus, Aliivibrio salmonicida, V. harveyi, V. tubiashii, and V. cholerae. Vibriosis, a disease which is caused by the abovementioned bacterial species, is a common and devastating bacterial disease in fish larviculture and aquaculture, and may be prevented by administering a composition, feed additive or animal feed as taught herein.

In an embodiment, the microbial infection is caused by a microbe of the species V. anguillarum, V. ordalii, V. vulnificus, V. alginolyticus, V. parahaemolyticus, Aliivibrio salmonicida, V. harveyi, V. tubiashii, and/or V. cholerae. In such case, the animal infected may be a fish or crustacean, e.g., a shrimp. In an embodiment, the microbial infection is caused by V. parahaemolyticus. The infected animal may be a crustacean, e.g., a shrimp.

In another embodiment, the microbial infection is caused by a Gram-positive bacterium, e.g. of the genus Enterococcus, e.g., Enterococcus cecorum.

Enterococci are commensals of the intestinal tract. Enterococcus species have natural resistance mechanisms to several classes of antimicrobials. In addition, Enterococcus readily acquires resistance to antimicrobials through mutations or acquisition of resistance genes. Enterococcus species Enterococcus, especially Enterococcus faecalis and Enterococcus faecium, is occasionally associated with mastitis in dairy animals. In poultry, enterococcal species like E. faecalis and E. cecorum can be involved in various infections such as arthritis and osteomyelitis.

In an embodiment, the microbial infection is caused by Enterococcus cecorum. Enterococcus cecorum was initially described in 1983 as a benign constituent of the gut microbiota of adult poultry, particularly chickens. However, over the past 15 years, pathogenic strains of E. cecorum have emerged as an important cause of skeletal disease in poultry, e.g., broiler and broiler breeder chickens. The most striking feature of infection with pathogenic E. cecorum is paralysis due to an inflammatory mass that develops in the spinal column at the level of the free thoracic vertebra. Recognition of this spinal lesion has given rise to several disease names for pathogenic E. cecorum infection which include vertebral osteomyelitis, vertebral enterococcal osteomyelitis and arthritis, enterococcal spondylitis and, colloquially, ‘kinky-back’. It must be noted that ‘kinky-back’ is also the common name for the developmental spinal anomaly, spondylolisthesis or enterococcocal spondylitis.

In another embodiment, the microbial infection is caused by a Gram-positive bacterium, e.g. of the genus Streptococcus, e.g., Streptococcus agalactiae.

Members of the genus Streptococcus cause mild to severe bacterial illnesses in animals. These organisms typically colonize one or more species as commensals and can cause opportunistic infections in those hosts. S. agalactiae is a common cause of subclinical mastitis in cattle. In some regions, it also seems to be fairly common in clinical or subclinical mastitis in sheep and goats. S. canis is an opportunistic pathogen that mainly affects dogs and cats. It can cause a variety of diseases including skin and soft tissue infections, arthritis, reproductive disease, mastitis, pneumonia, septicemia and streptococcal toxic shock-like syndrome, as well as cervical lymphadenitis in 3-6 month old kittens and otitis externa in dogs. In cats, this organism sometimes causes neonatal septicemia. S. dysgalactiae subsp. dysgalactiae is usually associated with clinical or subclinical mastitis in cattle, but it has been detected in other conditions in this species, including severe cellulitis and toxic shock-like syndrome. Some reports of S. dysgalactiae subsp. dysgalactiae in other hosts described mastitis in sheep and goats, suppurative polyarthritis in lambs, septicemia in fish and dogs, and septicemia and encephalitis in vampire bats.

In an embodiment, the bacterium of the genus Streptococcus is selected from the group consisting of Streptococcus agalactiae, Streptococcus canis, Streptococcus dysgalactiae subsp. Dysgalactiae, Streptococcus equi subsp. Zooepidemicus, Streptococcus halichoeri, Streptococcus iniae, and Streptococcus suis.

Non-limiting examples of viruses that may cause the microbial infection include, depending on the type of farming animal or companion animal, white spot syndrome virus (WSSV) causing white spot syndrome or white spot disease, Infectious Hypodermal and Hematopoietic Necrosis Virus (IHHNV) causing Infectious Hypodermal and Hematopoietic Necrosis, Taura Syndrome Virus (TSV) causing Taura Syndrome, Infectious Myonecrosis Virus (Myo/IMNV) causing Infectious Myonecrosis, Piscine myocarditis virus (PMCV) causing Piscine myocarditis, viral haemorrhagic septicaemia virus causing viral haemorrhagic septicaemia, infectious haematopoietic necrosis virus causing infectious haematopoietic necrosis, infectious salmon anaemia virus (ISAV) causing infectious salmon anaemia, piscine orthoreovirus (PRV) causing Heart and Skeletal Muscle Inflammation (HSMI), jaundice syndrome, proliferative darkening syndrome and erythrocytic body inclusion syndrome in fish, Tilapia lake virus, Covert mortality nodavirus, Shrimp hemocyte iridescent virus, and Abalone herpesvirus, Porcine reproductive and respiratory syndrome virus (PRRSV) causing Porcine reproductive and respiratory syndrome, porcine epidemic diarrhea virus (PEDV) causing porcine epidemic diarrhea, African swine fever virus causing, African swine fever, Classical swine fever virus causing Classical swine fever, Nipah virus, Swine vesicular disease virus causing swine vesicular disease, Transmissible gastroenteritis virus of swine causing transmissible gastroenteritis, avian influenza viruses causing avian influenza, Infectious bursal disease virus causing infectious bursal disease, Marek's disease virus causing Marek's disease, Newcastle disease virus causing Newcastle disease, Avian metapneumovirus, Avian infectious bronchitis virus causing avian infectious bronchitis, Infectious laryngotracheitis virus causing infectious laryngotracheitis, Duck hepatitis virus causing duck hepatitis, Pseudorabies virus causing pseudorabies, Bluetongue virus causing bluetongue virus, Foot-and-mouth disease virus (serotypes A, O, C, SAT1, SAT2, SAT3, Asia1) causing Foot-and-mouth disease, Japanese encephalitis virus causing Japanese encephalitis, Rabies virus causing rabies, Rift Valley fever virus causing Rift Valley fever, Rinderpest virus causing Rinderpest, Vesicular stomatitis virus causing Vesicular stomatitis, West Nile fever virus causing West Nile fever, and others. In an embodiment, the microbial infection is caused by a parasite, e.g., an endoparasite or an ectoparasite. The parasite may be a member of the phylum Apicomplexa that includes Cryptosporidium spp., Plasmodium spp., Eimeria spp., Neospora, Babesia, and Theileria.

Non-limiting examples of parasites taught herein include those of the genus Eimeria causing coccidiosis in a variety of animal species, those of the genus Cryptosporidium causing cryptosporidiosis, and those of the genus Enterocytozoon causing, a.o., early mortality syndrome (EMS) or acute hepatopancreatic necrosis disease (AHPND) in shrimp (Enterocytozoon hepatopenaei).

Parasites of the genus Eimeria include, without limitation, Eimeria tenella, Eimeria acervulina, Eimeria praecox, Eimeria mitis, Eimeria necatrix and Eimeria maxima causing coccidiosis in poultry, Eimeria zuernii, Eimeria alabamensis and Eimeria bovis causing coccidiosis in ruminants such as cattle, as well as any other Eimeria species that cause coccidiosis in various animal species.

Parasites of the genus Cryptosporidium include, without limitation, Cryptosporidium parvum, Cryptosporidium meleagridis, Cryptosporidium felis, Cryptosporidium canis and Cryptosporidium hominis all causing cryptosporidiosis.

In an aspect, the present disclosure provides a method of preventing, reducing the frequency and/or severity of, and/or treating any of the diseases mentioned herein, particularly in relation to the causative microbe.

The infected animal, or the animal at risk of infection may be an aquatic species, e.g., a fish or a shrimp.

In an embodiment, the present disclosure provides a method of preventing and/or treating Flavobacteriosis, Vibriosis, Enterococcosis or coccidiosis, and/or Streptococcosis in an animal, said method comprising the step of administering to said animal a composition, feed additive, or animal feed as taught herein. Streptococcosis is a general name for a variety of diseases caused by bacteria of the genus Streptococcus, e.g., those bacteria referred to herein.

The composition, animal feed and/or feed additive for use in the above methods may be as described hereinabove.

The extract may be as described hereinabove.

The animal may be a monogastric animal, a pre-ruminant animal, or a ruminant animal. The animals may be selected from farming animals and companion animals. The farming animals may be selected from the group consisting of poultry, swine, ruminants, e.g., beef cattle and dairy cattle, fish, e.g. salmon, trout, seabream, sea brass, tilapia, tuna, and the like, and crustaceans, e.g., shrimp. The companion animals may be selected from the group consisting of ornamental fish, cats, dogs, horses, rabbits, guinea pigs, and hamsters.

In an embodiment, the benefits, e.g., in performance, obtained in animals fed the feed additive or animal feed taught herein are relative to results obtained animals (of the same species) fed an identical animal feed yet without such feed additive.

The present invention is further illustrated, but not limited, by the following examples. From the above discussion and the examples, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### BRIEF DESCRIPTION OF THE FIGURE

The FIGURE shows the feed conversion ratio not adjusted (FCR) and adjusted for a standard 42 days old body weight (BW42 adjusted FCR) for the control treatment, Treatment 1 (control treatment+117 g per metric ton of feed of Glycyrrhiza glabra leaves) and Treatment 2 (control treatment+450 g per metric ton of a combination of 66% of Artemisia vulgaris aerial parts, 26% of Glycyrrhiza glabra leaves and 8% of Satureja hortensis aerial parts).

### EXAMPLES

### Example 1

This example describes that mixtures of Satureja hortensis (SH) aerial parts, Artemisia vulgaris (AV) aerial parts and Glycyrrhiza glabra (GG) aerial parts, in particular GG leaves, show a synergistic response on the control of inflammation in vitro.

Interleukin 6 (IL6) expression was used as a model for inflammation. The anti-inflammatory effect of mixtures of SH, AV, and GG was determined using the THP-1 human monocytic cell line which was differentiated into macrophage-like cells by phorbol myristate acetate. Prior to the assay the potential cytotoxic effect of the test compounds was determined using the MTT (3-(4,5-dimethylthiazol-2-yl)-2,5-diphenyltetrazolium bromide) assay for determining mitochondrial dehydrogenase activities in the living cells. The anti-inflammatory effect on cytokine release in the THP-1 macrophage cells was measured by incubating the cells with the treatments followed by the addition of LPS to stimulate the cytokine production. After 24 h the amount of IL6 was determined in the supernatant of the cell cultures by capture ELISA using commercial kits.

The 3 plants and plant parts involved in the study were aerial part of Satureja hortensis (SH), aerial parts of Artemisia vulgaris (AV) and leaves of Glycyrrhiza glabra (GG). The raw materials came either from cultivation or from wild crafting. Cultivation or wild crafting and drying followed Good Agricultural and Collecting practices. Dry arial parts for Satureja hortensis and Artemisia vulgaris and dry leaves for Glycyrrhiza glabra were ground to a powder having a particle size of <0.3 mm.

Satureja hortensis and Artemisia vulgaris powder were extracted using 70% m/m ethanol, whereas Glycyrrhiza glabra leaf powder was extracted using 30% m/m ethanol. Plant material:solvent ratio was 1:10. Extraction of the plant powder was carried out at 40° C. for 60 min under continuous stirring. The raw extracts were filtered under vacuum using a glass-fiber filter. The fluid extracts were then concentrated to dryness in a rotation evaporator at 40° C. under reduced pressure (100 mbar). The dry residue was dissolved in 70% m/m ethanol and the sterile filtered. Of all extracts the dry matter content was determined. These extracts were then mixed according to the mixture design scheme set forth in the FIGURE.

The study used a simplex extreme vertices design as shown by the FIGURE. This type of experimental design was used to determine whether mixtures of different components could yield a synergistic effect. SH, AV and GG are the concentration (in mg/L) of the plant extracts of SH, AV and GG in the 250 mg/L total mixture. Additional treatments were also considered: a control, 125 mg/L AV, 100 mg/L GG. All treatments were triplicated.

The results for IL6 expressed in the THP-1 human monocytic cell line in pg/mL were analyzed using the following full cubic model Y=SH+AV+GG+SH×AV+SH×GG+AV×GG+SH×AV×GG+ε, with stepwise of α=15%. This model captures the linear effect, the nonlinear synergistic and antagonistic binary effect, and the nonlinear synergistic and antagonistic ternary effect. In this model, Y is the IL6 response in the THP-1 human monocytic cell line expressed in pg/mL, ε is the residual error of the model, SH, AV and GG are the concentration (in mg/L) of the plants SH, AV and GG in the 250 mg/L total mixture.

Table 1 summarizes the basic statistics of the IL6 outcome for the mixture design.

TABLE 1
Basic statistics of the IL6 expression, expressed in pg/mL, for the Treatment type.

| | Treatments | | | | | | | | | | | |
|---|---|---|---|---|---|---|---|---|---|---|---|---|
| | Plant amounts, in mg/L | | | Plants proportions, in % | | | Basic statistics for IL-6, in pg/mL | | | | | |
| Treatment type | SH | AV | GG | SH | AV | GG | N | Mean | SEM | Minimum | Median | Maximum |
| Control | 0.00 | 0.00 | 0.00 | | | | 3 | 10203 | 1037 | 8866 | 9497 | 12245 |
| GG | 0.00 | 0.00 | 100.00 | 0% | 0% | 100% | 3 | 1848 | 133 | 1712 | 1718 | 2113 |
| AV | 0.00 | 125.00 | 0.00 | 0% | 100% | 0% | 3 | 3279 | 249 | 2945 | 3126 | 3766 |
| SH | 250.00 | 0.00 | 0.00 | 100% | 0% | 0% | 3 | 1247 | 118 | 1031 | 1275 | 1436 |
| SH + AV (50:50) | 125.00 | 125.00 | 0.00 | 50% | 50% | 0% | 3 | 1583 | 175 | 1238 | 1709 | 1803 |
| SH + AV (75:25) | 187.50 | 62.50 | 0.00 | 75% | 25% | 0% | 3 | 1861.1 | 34.0 | 1802.1 | 1861.1 | 1920.0 |
| SH + GG (80:20) | 200.00 | 0.00 | 50.00 | 80% | 0% | 20% | 3 | 1045.5 | 50.1 | 963.0 | 1037.5 | 1136.0 |
| SH + GG (60:40) | 150.00 | 0.00 | 100.00 | 60% | 0% | 40% | 3 | 322.5 | 71.0 | 187.6 | 351.5 | 428.3 |
| SH + AV + GG (10:50:40) | 25.00 | 125.00 | 100.00 | 10% | 50% | 40% | 3 | 95.0 | 55.3 | 1.2 | 91.2 | 192.6 |
| SH + AV + GG (30:50:20) | 75.00 | 125.00 | 50.00 | 30% | 50% | 20% | 3 | 577.4 | 50.2 | 481.9 | 597.9 | 652.3 |
| SH + AV + GG (53:38:10) | 131.25 | 93.75 | 25.00 | 53% | 38% | 10% | 3 | 885.5 | 30.9 | 823.8 | 912.7 | 920.1 |
| SH + AV + GG (58:13:30) | 143.75 | 31.25 | 75.00 | 58% | 13% | 30% | 3 | 359.5 | 76.4 | 214.9 | 389.1 | 474.6 |
| SH + AV + GG (35:25:40) | 87.50 | 62.50 | 100.00 | 35% | 25% | 40% | 3 | 549.6 | 87.5 | 445.3 | 479.9 | 723.5 |
| SH + AV + GG (55:25:20) | 137.50 | 62.50 | 50.00 | 55% | 25% | 20% | 3 | 1086.2 | 7.07 | 1072.1 | 1092.6 | 1094.0 |
| SH + AV + GG (78:13:10) | 193.75 | 31.25 | 25.00 | 78% | 13% | 10% | 3 | 1114.4 | 66.9 | 1004.9 | 1102.4 | 1235.7 |
| SH + AV + GG (32:38:30) | 81.25 | 93.75 | 75.00 | 32% | 38% | 30% | 3 | 458.0 | 51.8 | 376.9 | 442.6 | 554.4 |

The line “Control” is the control without addition of plants. All treatments are denoted in the first column. In the table, “SH” stands for Satureja hortensis aerial parts extract, “AV” for Artemisia vulgaris aerial parts extract, and “GG” for Glycyrrhiza glabra leaves extract. The figures in the columns entitled “treatments”, “plant amounts, in mg/L” are the amounts of each plant extract expressed in mg/mL in the 250 mg/L total mixture. The figures in the columns entitled “treatments”, “plants proportions in %” are the proportion of each plant extract expressed in the total mixture. N is the number of replicates per treatment, SEM the Standard Error of the mean.

From Table 1, it can be concluded that extracts of Satureja hortensis aerial parts, Artemisia vulgaris aerial parts, and Glycyrrhiza glabra leaves individually reduce IL6 expression, confirming that each of the plants individually has anti-inflammatory activity. Moreover, Table 1 shows that Glycyrrhiza glabra leaves extract and Satureja hortensis aerial parts extract acted synergistically to further decrease IL6 production. Moreover, the combination of the 3 plants showed a synergistic effect to even further decrease the production 15 of IL6.

### Example 2

The effect of dietary inclusion of dried Glycyrrhiza glabra leaves and a mixture of Satureja hortensis aerial parts, Artemisia vulgaris aerial parts and Glycyrrhiza glabra leaves on the productive performance of broiler chicken was investigated.

Satureja hortensis aerial parts, Artemisia vulgaris aerial parts and Glycyrrhiza glabra leaves came from cultivation. Cultivation and drying followed Good Agricultural and Collecting practices. The material for the 3 plants were ground to a particle size of <0.3 mm.

The 3 plants were combined in a mixture with the following proportion: 66% of Artemisia vulgaris aerial parts, 26% of Glycyrrhiza glabra leaves and 8% of Satureja hortensis aerial parts (SH+AV+GG (8:66:26)).

Diets, fed in mash form, were provided by age of the birds: Starter feed was fed from 0 day to 14 days, Grower feed was fed from 15 days to 28 days and Finisher feed was fed from 29 days to 42 days. The basal diets compositions were the following:

| | Starter | Grower | Finisher |
|---|---|---|---|
| Ingredients, in kg/100 kg | feed | feed | feed |
| Corn | 58.44 | 60.36 | 64.56 |
| Soybean meal | 30.469 | 23.82 | 19.87 |
| distiller's dried grains with solubles | 4.00 | 8.00 | 8.00 |
| meat and bone meal | 4.00 | 4.00 | 3.821 |
| soybean oil | 0.972 | 2.024 | 2.256 |
| Limestone | 0.309 | 0.287 | 0.221 |
| dicalcium phosphate | 0.609 | 0.281 | 0.110 |
| Salt | 0.391 | 0.375 | 0.379 |
| Choline | 0.064 | 0.069 | 0.069 |
| Lysine | 0.218 | 0.273 | 0.259 |
| Methionine | 0.287 | 0.266 | 0.236 |
| Threonine | 0.107 | 0.106 | 0.085 |
| Mixture supplying other minerals and | 0.143 | 0.143 | 0.143 |
| vitamins | | | |

The experimental treatments were made as follow. The Control treatment birds received basal Starter feed, basal Grower feed and basal Finisher feed. Treatment 1 birds received the basal diets, all supplemented with 117 g per metric ton (117 ppm) of feed of dried ground Glycyrrhiza glabra leaves. Treatment 2 birds received the basal diets, all supplemented with 450 g per metric ton (450 ppm) of feed of the product containing 66% of Artemisia vulgaris aerial parts, 26% of Glycyrrhiza glabra leaves and 8% of Satureja hortensis aerial parts (SH+AV+GG (8:66:26)) defined above. Treatment 1 and Treatment 2 both supplied the same amount of Glycyrrhiza glabra leaves. Hence, the comparison of both treatments will show if there is a synergistic effect of the 2 other ingredients of the mixture.

Each experiment treatment was assigned to 12 pens per treatment. Each pen (dimensions of each pen are 4′×5′) contained 1 water fountain and a feed tube. Birds started on new wood shavings. 34 Ross 308 day 0 chicks were allocated to each pean. Birds were distributed to pens on day 1 so that equal distribution of birds by body weight among the 108 study pens. Continuous lighting was provided Od to 42 days.

All broilers were weighed by pen when bird ages were 0, 14, 28 and 42d. Feed was weighed in as needed and weighed out when birds were weighed. The following data were collected during the study: broiler body weights and feed intakes at day 42. Mortality, feed conversion and mortality at day 42. Based on these outcomes, feed conversion and adjusted feed conversion were calculated.

Feed conversion was calculated for a pen as the total feed consumed divided by (sum of total of all body weight for the pen+sum total of all mortality weight for the pen).

Adjusted feed conversion ratio with final BW adjustment at day 42 was calculated as follows: Feed conversion+(2.22 kg−actual body weight)/7.

The statistical analysis was a one-way ANOVA with the following model Yᵢⱼ=μ+Treatmentᵢ+εᵢⱼ in which Y is the productive response which average is μ, Treatment is a categorical variable with 3 levels (Control or Treatment 1 or Treatment 2) and ε is the residual error. Means for the 3 levels of the Treatment were compared using the Fisher test with 95% confidence.

The results are presented in the FIGURE. The FIGURE shows that the addition of Glycyrrhiza glabra ground leaves in the diet of broiler chicken (Treatment 1) improved productive performance at day 42. Moreover, the addition of Glycyrrhiza glabra ground leaves improved bodyweight-adjusted FCR by 2.1% compared to the control. The addition of a mixture of Satureja hortensis aerial parts, Artemisia vulgaris aerial parts and Glycyrrhiza glabra leaves (Treatment 2) provided an additional 3.5% improvement of bodyweight-adjusted FCR (P<0.05) demonstrating that the mixture of Satureja hortensis aerial parts, Artemisia vulgaris aerial parts and Glycyrrhiza glabra leaves showed a synergistic improvement over the addition of Glycyrrhiza glabra ground leaves alone (Treatment 1).

### Example 3

The effect of a composition comprising Glycyrrhiza glabra leaves, Satureja hortensis aerial parts and Artemisia vulgaris aerial parts (same as in Example 2) on the productive performance of shrimps was investigated.

Glycyrrhiza glabra leaves, Satureja hortensis aerial parts and Artemisia vulgaris aerial parts came from cultivation. Cultivation and drying followed Good Agricultural and Collecting practices. The Glycyrrhiza glabra leaves were grinded to a particle size of <0.3 mm. The basal diet composition was as set forth in Table 2.

TABLE 2
Basal diet composition of extruded shrimp feed.

| Ingredients | Inclusion levels (in kg/100 kg) |
|---|---|
| Soya products/by-product | 35-40 |
| Wheat products/by-product | 35-40 |
| Fish meal/by-product | 8-15 |
| Macro mineral | 4-6 |
| Soy Lecithin/oil | 1.5-2.5 |
| Fish oil NA | 1.5-2 |
| Crustacean-based attractant | 1.5-3 |
| Hydrolysates | 1.5-3 |
| Amino acids mix | 0.5-1.2 |
| Vitamin-mineral premix | 0.3-0.5 |

The diets were produced as 1.4±0.1 mm extruded pellets.

The feed production procedure followed the normal guidelines for shrimp feed extrusion. First, all dried raw materials (except micro-ingredients) were ground through a 1 mm (50 Hz) grinder screen. Next, ingredients were weighed to a 0.01-g precision on an electronic scale following the formula specifications. After the extrusion, the pellets were dried until the moisture content was below 10%. Final feeds were bagged and stored at 4° C.

The experimental treatments were made as follow. The ‘Control’ shrimps received basal shrimp feed. ‘Treatment’ shrimps received the basal diet supplemented with 2020 g per metric ton of feed (2020 ppm) of a composition comprising dried Glycyrrhiza glabra leaves, dried Satureja hortensis aerial parts and dried Artemisia vulgaris aerial parts.

The Control and the Treatment were each assigned to 1 plastic tank. Each tank of a capacity of 290 L was equipped with an individual filtration system and filled with artificial seawater at a salinity of 20 g/L. 75 shrimps were introduced in each tank. Shrimps were Penaeus vannamei post larvae.

The total weight of the groups was taken at the beginning (day 0) and at the end of the trial (day 18). The mean body weight of the shrimps was calculated at the beginning and at the end for each group. The daily feeding rate for each group was calculated based on shrimp mean body weight and adjusted daily according to the expected shrimp growth and mortality. Feed was distributed automatically 6 times a day during a period of 18 days. Water quality was maintained by the filtration system and regular water changes. Water temperature was kept at 24° C. The room in which the tanks were located was illuminated 12 h per day.

Growth performance was assessed by the following parameters: weight gain (WG) and feed conversion ratio (FCR).

TABLE 3
Performance of shrimps

| Productive response | | | |
|---|---|---|---|
| (unit) | Control | Treatment | Effect of treatment (%) |
| Final weight, g | 2.73 | 2.87 | +5% |
| Cumulative weight gain, g | 2.13 | 2.25 | +6% |
| FCR | 0.95 | 0.92 | −4% |

The results presented in Table 3 show that the addition of the composition comprising dried Glycyrrhiza glabra leaves, dried Satureja hortensis aerial parts and dried Artemisia vulgaris aerial parts in the diet of shrimp improved productive performance. The addition of said composition increased weight gain by 6%. Feed conversion ratio was decreased (which means feed efficiency was improved) by the addition of said composition by 4%.

### Example 4

In this experiment the effect of a composition comprising dried Glycyrrhiza glabra leaves, dried Satureja hortensis aerial parts and dried Artemisia vulgaris aerial parts (same as in Example 2) on the response of shrimps to a disease challenge was investigated. The disease challenge was an acute hepatopancreatic necrosis disease (AHPND) challenge produced by the bacteria Vibrio parahaemolyticus.

Glycyrrhiza glabra leaves, Satureja hortensis aerial parts and Artemisia vulgaris aerial parts came from cultivation. Cultivation and drying followed Good Agricultural and Collecting practices. The Glycyrrhiza glabra leaves were grinded to a particle size of <0.3 mm. The composition of the basal diet is shown in Table 4 below.

TABLE 4
Basal diet composition of extruded shrimp feed.

| Ingredients | Inclusion levels (in kg/100 kg) |
|---|---|
| Soya products/by-product | 35-40 |
| Wheat products/by-product | 35-40 |
| Fish meal/by-product | 8-15 |
| Macro mineral | 4-6 |
| Soy Lecithin/oil | 1.5-2.5 |
| Fish oil NA | 1.5-2 |
| Crustacean-based attractant | 1.5-3 |
| Hydrolysates | 1.5-3 |
| Amino acids mix | 0.5-1.2 |
| Vitamin-mineral premix | 0.3-0.5 |

The diets were produced as 1.4±0.1 mm extruded pellets.

The feed production procedure followed the normal guidelines for shrimp feed extrusion. First, all dried raw materials (except micro-ingredients) were ground through a 1 mm (50 Hz) grinder screen. Next, ingredients were weighed to a 0.01-g precision on an electronic scale following the formula specifications. After the extrusion, the pellets were dried until the moisture content was below 10%. Final feeds were bagged and stored at 4° C.

During the first part of the experiment, the shrimps were housed in groups and fed either the “Control” diet or the “Treatment” diet. These 2 diets were obtained as follow: the Control group received the basal diet; the Treatment group received the control diet supplemented with 4040 g of a composition comprising dried Glycyrrhiza glabra leaves, dried Satureja hortensis aerial parts and dried Artemisia vulgaris aerial parts per metric ton of feed (at 4040 ppm).

The Control and the Treatment groups were each assigned to 1 plastic tank. Each tank of a capacity of 290 L was equipped with an individual filtration system and filled with artificial seawater at a salinity of 20 g/L. 75 shrimps were introduced in each tank. Shrimps were Penaeus vannamei post larvae.

After 18 days of growth, the shrimps from these 2 groups were transferred to the disease challenge facility and housed in the infection unit (1 shrimp per tank) for acclimatization. Animals were housed individually in 10 L tanks and fed with the same diet they were fed during the first 18 days. Tanks were filled with artificial seawater with salinity of 20 g/L and equipped with mechanical/biological filter that ensured water quality. Water temperature was kept at 27° C. and the room in which the tanks were kept was illuminated 12 h a day. They were allowed to acclimatize for 3 days.

Each treatment (Control or Treatment) replicates correspond to 3 blocks of 10 individual shrimp per treatment. Shrimps were fed manually 4 times a day (9 am, 11:30 am, 2 μm, 4:30 μm) and additional feeding was provided at 8 μm and 12 μm on the days of AHPND inoculation. Shrimps were monitored twice daily for clinical signs of disease and mortality.

The challenge was an acute hepatopancreatic necrosis disease (AHPND) challenge. The bacterium used was a Vibrio parahaemolyticus isolated from shrimp suffering from AHPND. A stock of this bacterium is permanently kept frozen at −70° C. After thawing, the stock was aseptically inoculated in culture medium and grown using standard conditions. The optical density of the resulting bacterial suspension was determined spectrophotometrically. These data were used to determine the concentration of bacteria in the suspension in colony forming units per millimeter. Quantified suspensions of the bacteria were used to inoculate shrimp by immersion in all phases of this experiment. Each tank was inoculated with the same dose of bacteria.

Mortality rate was measured and cumulative mortality at 14 days post infection in (%) was calculated. The results are shown in Table 5 below.

TABLE 5
Mortality of shrimp subjected to Vibrio parahaemolyticus infection.

| | Cumulative mortality at |
|---|---|
| | 14 days post infection |
| | (%) |
| Control | 59.5% |
| Treatment | 40% |
| Effect of Treatment vs Control (%) | —33% |

The addition of a composition comprising Glycyrrhiza glabra leaves, Satureja hortensis aerial parts and Artemisia vulgaris aerial parts to the diet of shrimps significantly reduced mortality due to Vibrio parahaemolyticus.

### Example 5

In this experiment the effect of a composition comprising Glycyrrhiza glabra leaves, Satureja hortensis aerial parts and Artemisia vulgaris aerial parts (same as in Example 2) on the response of broilers to an Enterococcus cecorum challenge was investigated.

Birds were hatched on-farm to reduce risk of hatchery contamination with E. cecorum. From Day 0 until the end of the study at day 14, 80 birds (ROSS 308 broilers) per treatment were neck-tagged and equally distributed over four pens (i.e., 20 birds per pen).

All animals in the Treatment (challenged; treatment with 200 ppm of a composition comprising Glycyrrhiza glabra leaves, Satureja hortensis aerial parts and Artemisia vulgaris aerial parts) and positive control (challenged; no treatment) group were challenged with 0.5 mL of a mix of pathogenic Enterococcus cecorum isolate via oral gavage (concentration: 1.65*108 colony forming units/mL). The birds from the negative control (non-challenged; no treatment) were sham-inoculated using the same volume (i.e. 0.5 mL per bird) of culture medium.

The trial was designed to mimic field conditions and therefore consisted of two phases after initial infection through oral gavage with E. cecorum at day 0 of life. Phase 1 consisted of 7 days of heat stress challenge (+4° C. above recommended levels in ROSS308 guidelines) and 24-hour delayed onset of feeding to increase translocation (mimic translocation levels encountered in the field), followed by Phase 2 consisting of 7 days in unchallenged conditions to observe recovery potential.

At day 7 and day 14, 10 chicks per pen were euthanized to collect the spleen and heart. The tissues were individually analysed using qPCR as described by Jung et al (2017; Bmc Microbiology 17 (1), 106), using a cut-off value of 36 to identify positive organs. Organs were analysed through qPCR and positive duplicates removed: an animal with either an infected spleen, heart or both was considered ‘infected’. The prevalence found at day 7 was considered the status at the end of the heat-stress challenge (7 days post-infection), whereas the prevalence at 14 days post-infection is the status after 7 days of recovery in unchallenged conditions.

### Results

Table 6 shows the percentage of chicks with Enterococcus cecorum infected organs at day 7 and day 14 post-challenge.

TABLE 6
Percentage of chicks with Enterococcus cecorum infected
organs at day 7 and day 14 post-challenge.

| | % chicks with EC | % chicks with EC |
|---|---|---|
| | infected organs d 7 | infected organs d 14 |
| | post-challenge | post-challenge |
| Non-challenged; non-treated | 3 | 0 |
| Challenged; non-treated | 73 | 33 |
| Challenged; treated with | 73 | 15 |
| SH + AV + GG (8:66:26) | | |

The high prevalence of infected organs at day 7 confirms a successful challenge with ample translocation of the pathogenic Enterococcus cecorum strain that was administered at birth (day 0). These findings are in line with the two other challenge models published (e.g. Borst et al. 2019 & Schreier et al. 2021). Animals receiving the Treatment SH+AV+GG (8:66:26) showed a high level of translocation at day 7 (73%) but were capable to reduce the prevalence of infected organs at Day 14 to 15%. Part of the recovery may be attributed to the animals' own immune response, whereas the results indicate that additional support for recovery may be attributed to the Treatment. The difference of 58 percent points is considered a relevant impact in mitigating production losses from E. cecorum infections in poultry.

### Example 6

Example 1 was repeated with various mixtures of Glycyrrhiza glabra leaves extract (GG) and Artemisia vulgaris extract (AV). The results are summarised in Table 7.

TABLE 7
Mean IL6 expression, expressed in pg/mL, per Treatment type.
All treatments are denoted in the first column. “Control”
is the control without addition of plants. In the table,
“AV” stands for Artemisia vulgaris aerial parts
extract, and “GG” for Glycyrrhiza glabra leaves extract.

| | Plant proportions (%) | | Mean IL-6 production |
|---|---|---|---|
| Treatment type | AV | GG | (pg/mL) |
| Control | 0 | 0 | 2472.1 |
| GG | 0 | 100 | 110.7 |
| AV | 100 | 0 | 1086.2 |
| AV + GG (50:50) | 50 | 50 | 125.3 |
| AV + GG (25:75) | 25 | 75 | 16.8 |
| AV + GG (75:25) | 75 | 25 | 462.8 |

Regression analysis also demonstrated that when combined, AV and GG extracts show synergistic anti-inflammatory effect, larger than the arithmetic addition of the individual effects of both plants (P-value<0.01).

1. A composition comprising (i) Glycyrrhiza glabra or an extract thereof, and at least one of (ii) Satureja hortensis or an extract thereof and (iii) Artemisia vulgaris or an extract thereof.

2. The composition of claim 1, wherein the Glycyrrhiza glabra comprises Glycyrrhiza glabra aerial parts or an extract thereof, and/or the Satureja hortensis comprises Satureja hortensis aerial parts or an extract thereof, and/or the Artemisia vulgaris comprises Artemisia vulgaris aerial parts or an extract thereof.

3. The composition according to claim 2, wherein the Glycyrrhiza glabra aerial parts comprises Glycyrrhiza glabra leaves.

4. The composition according to claim 2, wherein the Satureja hortensis aerial parts, and/or the Artemisia vulgaris aerial parts, and/or the Glycyrrhiza glabra aerial parts are dried, and optionally ground.

5. The composition according to claim 1, wherein the extract is selected from an aqueous extract, ethanolic extract, methanolic extract, isopropanolic extract, ethyl acetate extract, acetonic extract, hexane extract, a supercritical CO2 extract, and combinations thereof.

6. An animal feed comprising a composition according to claim 1.

7. The animal feed according to claim 6, wherein the composition is present in an amount of about 5 to about 10000 mg/kg feed.

8. A method of improving performance in an animal, the method comprising administering to the animal an animal feed according to claim 6, wherein improvement is measured relative to results obtained for an animal of the same species fed an identical animal feed but without the composition according to claim 1.

9. The method according to claim 8, wherein the improved performance comprises increasing feed intake, increasing average daily gain, and/or increasing feed efficiency.

10. The method according to claim 8, wherein the animal is a farming animal or companion animal.

11. A method of decreasing IL6 levels in an animal, the method comprising administering to the animal a composition according to claim 1.

12. A method of preventing and/or reducing inflammation in an animal, the method comprising administering to the animal a composition according to claim 1.

13. The method according to claim 12, wherein the animal is a farming animal or companion animal.

14. A method of preventing and/or reducing microbial infection and/or diseases caused by such microbial infection in an animal, the method comprising administering to the animal a composition according to claim 1.

15. The method according to claim 14, wherein the microbe is selected from a Gram-negative and a Gram-positive bacterium.

16. The method according to claim 14, wherein the microbe is a Vibrio species, such as Vibrio parahaemolyticus.

17. The method according to claim 14, wherein the microbe is an Enterococcus species, such as Enterococcus cecorum.

18. The method according to claim 14, wherein the animal is a farming animal or companion animal.

19. The method according to claim 18, wherein the animal is a farming animal selected from poultry, swine, ruminants, e.g., beef cattle and dairy cattle, fish, e.g. salmon, trout, seabream, sea brass, tilapia, tuna, and the like, and crustaceans, e.g., shrimp.

20. The method according to claim 18, wherein the animal is a companion animal selected ornamental fish, cats, dogs, horses, rabbits, guinea pigs, and hamsters.
